(19) United States
(12) Patent Application Publication
(10) Pub. No.: US 2012/0059036 A1
(43) Pub. Date: Mar. 8, 2012

(54) METHOD FOR REDUCING THROMBOCYTOPENIA AND THROMBOCYTOPENIA-ASSOCIATED MORTALITY

(76) Inventor: Marco Valgimigli, Ferrara (IT)

(21) Appl. No.: 13/130,340

(22) PCT Filed: Nov. 20, 2009
(86) PCT No.: PCT/US2009/006240

§ 371 Date: Nov. 4, 2011

Related U.S. Application Data
(60) Provisional application No. 61/116,976, filed on Nov. 21, 2008.

(51) Int. Cl. A61K 31/4465; A61P 7/00

(52) U.S. Cl. 514/331

(57) ABSTRACT

Disclosed are methods for reducing the risk of thrombocytopenia-associated mortality and morbidity, and for reducing the risk of becoming thrombocytopenic, in patients whose treatment requires inhibition of platelet aggregation. The methods involve administration of a pharmaceutically acceptable salt of tirofiban.

### BACKGROUND OF THE INVENTION

Platelet reactivity (i.e., activation and aggregation) is pivotal in the pathogenesis of complications after percutaneous coronary intervention (PCI) and the degree of platelet inhibition during and immediately after PCI is critical for protecting against further ischemic events. Such events include reinfarction, reocclusion of the target vessel and other vaso-occlusive disorders. Such events can occur spontaneously or in response to an invasive cardiac procedure, such as PCI, coronary artery or peripheral bypass grafting and cardiac valve replacement.

Historically, many measures have been taken to inhibit platelet aggregation. Among these measures is the intravenous administration of inhibitors of the glycoprotein (GP) IIb/IIIa receptor complex. These inhibitors include abciximab, tirofiban and eptifibatide. These inhibitors should be used concomitantly with treatments known to trigger unwanted platelet aggregation (e.g., administration of unfractionated heparin). However, it has also been widely observed that there are inherent risks associated with the administration of GP IIb/IIIa inhibitors. These risks include major and minor bleeding and, of particular concern, onset of thrombocytopenia. It has been observed, in fact, that some patients who are spared reinfarction and even death following PCI can instead suffer from, and even succumb to, the effects (primarily gastrointestinal or cranial bleeding) of the thrombocytopenia induced by treatment with platelet-aggregation inhibitors.

In patients with ST-segment elevation myocardial infarction (STEMI), platelet reactivity is associated with the severity of myocardial damage¹ and strongly correlates with various measures of myocardial reperfusion, including ST-segment recovery after treatment.^{2,3} In a recent study of abciximab vs placebo in patients undergoing primary angioplasty, the degree of ST-segment resolution was significantly improved with abciximab,⁴ as was the mortality rate at 12 months.⁵ Tirofiban belongs to the same class of anti-platelet agents as abciximab, namely glycoprotein IIb/IIIa inhibitors. However, tirofiban differs from abciximab in terms of both pharmacodynamic and pharmacokinetic profiles.⁶

Similar to abciximab, tirofiban inhibits platelet activity through glycoprotein IIb/IIIa platelet receptor blockade, but unlike abciximab, tirofiban exerts a competitive and rapidly reversible antagonism and does not inhibit other β3 integrins, such as the vitronectin receptor, at the surface of vascular cells or the activated Mac-1 receptor on leukocytes.⁷ These have traditionally been regarded as crucial targets to explain abciximab effects especially on microcirculation in the setting of ongoing myocardial infarction.⁸

The first head-to-head comparison between abciximab and tirofiban was powered based on the preservation of a difference of at least 50% in the effect of abciximab as compared with that of placebo⁹. In that study, abciximab was superior to tirofiban with respect to the prespecified combined end point.⁹ This result was driven by a higher rate of periprocedural myocardial infarction in the tirofiban group, suggesting inadequate early platelet inhibition with the bolus regimen (10 μg/kg) used.⁹ Subsequent dose-ranging studies showed that increasing the tirofiban bolus dose from 10 to 25 μg/kg provided an optimal level of platelet inhibition,¹⁰ and several independent pharmacokinetics studies suggested that tirofiban, at increased dose, might even lead to a more consistent platelet inhibition than abciximab.¹¹⁻¹³ To date, three small single-center investigations^{11, 14, 15} and one prematurely-stopped multicenter randomized study¹⁶ have compared high-dose tirofiban with abciximab in 719 patients undergoing PCI; however, none of these studies had adequate power to evaluate the comparison between the two drugs.

There is a need for a treatment regimen that has the desired effect of inhibiting platelet aggregation but in which there is at the same time a reduction of thrombocytopenia and thrombocytopenia-associated mortality, particularly in those patients susceptible to thrombocytopenia.

Induction of thrombocytopenia following administration of tirofiban has been observed, but not in significantly higher amounts compared to placebo or null-treatment arm. It has widely been thought that tirofiban, as a GP IIb/IIIa receptor antagonist, would have a comparable risk profile to the other drugs in the class.

### BRIEF SUMMARY OF THE INVENTION

The present invention is the discovery that, surprisingly, a high-dose bolus (HDB) of tirofiban hydrochloride followed by a continuous infusion of tirofiban hydrochloride over a number of hours results in significantly reduced incidence of both thrombocytopenia and thrombocytopenia-associated morbidity and mortality compared to the effects of abciximab.

### BRIEF DESCRIPTION OF THE DRAWINGS

FIG. 1 shows the results of noninferiority analysis of tirofiban when compared to abciximab.

FIG. 2 shows the effect of thrombocytopenia on patient outcome in patients subjected to a primary PCI procedure.

FIG. 3 shows the results of comparison of the impact of thrombocytopenia on mortality in patients treated with HDB tirofiban or abciximab.

FIG. 4 shows the results of comparison of the likelihood of patients experiencing a clinical event (death or myocardial infarction) within eight months of treatment with HDB tirofiban or abciximab.

### DETAILED DESCRIPTION OF THE INVENTION

Tirofiban hydrochloride, commercially available as AGGRASTAT®, is a nonpeptide inhibitor of the platelet GP IIb/IIIa receptor, the major platelet surface receptor involved in platelet aggregation. It is chemically described either as N-(butylsulfonyl)-O-[4-(4-piperidinyl)butyl]-tyrosine monohydrochloride or 2-S-(n-butylsulfonylamino)-3[4-(piperidin-4-yl)butyloxyphenyl]propionic acid hydrochloride and is described in U.S. Pat. No. 5,292,756. Its structure is:

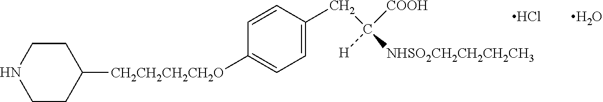

From October 2004 to April 2007, a Phase III, open-label, multinational study of 745 patients experiencing ST-segment elevation myocardial infarction (STEMI), entitled the Multicentre Evaluation of Single High-Dose Bolus Tirofiban vs Abciximab With Sirolimus-Eluting Stent or Bare Metal Stent in Acute Myocardial Infarction Study (MULTISTRATEGY) was conducted. The basic design of the study was detailed earlier.¹⁷ Briefly, patients were randomly assigned with the use of a 2×2 factorial design to one of four interventional strategies of reperfusion: abciximab with an uncoated stent; abciximab with a sirolimus-eluting stent; HDB tirofiban hydrochloride with an uncoated stent; or HDB tirofiban hydrochloride with a sirolimus-eluting stent. Patient characteristics were similar among all four groups except that there was a slightly higher prevalence of prior transient ischemic attacks in the tirofiban/uncoated-stent groups. The inclusion criteria were (1) chest pain for longer than 30 minutes with an electrocardiographic ST-segment elevation of 1 mm or greater in two or more contiguous electrocardiogram leads, or with a new left bundle-branch block, and (2) admission either within 12 hours of symptom onset or between 12 and 24 hours after onset with evidence of continuing ischemia. The exclusion criteria included administration of fibrinolytics in the previous 30 days, major surgery within 15 days, and active bleeding or previous stroke in the last six months. Immediately after eligibility criteria were met and before the visualization of coronary arteries through angiography, the treating physician at each investigational site performed open-label assignments of study treatments via sealed envelopes. Randomization was achieved with a 1:1:1:1 computer-generated random sequence supplied by an academic statistician, without stratification, in blocks of 30.

Tirofiban hydrochloride was administered in a high-dose bolus (25 μg/kg bolus) followed by a continuous infusion (0.15 μg/kg/min for 18-24 hours). This type of regimen is described in U.S. Pat. No. 6,770,660. Abciximab was administered in a 0.25 mg/kg bolus, followed by 0.125 μg/kg/min continuous infusion for 12 hours. The administration of both drugs began at first medical contact, before arterial sheath insertion. Heparin was given at 40 to 70 U/kg, targeting an activated clotting time of at least 200 seconds. Patients received aspirin (160-325 mg orally or 250 mg intravenously, followed by 80-125 mg/d orally indefinitely) and clopidogrel (300 mg orally and then 75 mg/d for at least three months).

A 12-lead electrocardiogram was recorded before the procedure and 90 minutes after the last balloon inflation in the infarct-related artery. Follow-up visits were scheduled at one, four, and eight months.

The data for all patients with primary end-point events were reviewed by an independent adjudication committee whose members were blinded to treatment assignments. Events adjudication was performed separately by two members, and in case of disagreement, the opinion of the third member was obtained and the final decision taken by consensus. The committee was also responsible for the adjudication of all clinical events according to the Academic Research Consortium.¹⁸

Changes in the ST-segment of the electrocardiogram were evaluated cumulatively before and 90 minutes after intervention. ST-segment elevation was measured to the nearest 0.5 mm at 60 milliseconds after the J point by a single experienced cardiologist who was blinded to treatment assignments. The intraobserver agreement was 94.1% (K=0.82) in identifying the recovery by at least 50% of ST-segment elevation in 217 randomly selected patients (30% of all interpretable electrocardiograms). Quantitative angiographic analyses were performed with a validated edge-detection system (CAAS II; Pie Medical, Maastricht, the Netherlands), and coronary flow was classified according to Thrombolysis in Myocardial Infarction (TIMI) criteria. Angiographic analyses and TIMI grading were performed by one independent cardiologist who was blinded to treatment assignments.

Discrete data were summarized as frequencies, and comparisons were made with the likelihood-ratio χ² test or Fisher exact test. Continuous data were expressed as mean (SD) or median and interquartile range according to their distribution; comparisons were made with a one-way analysis of variance or the Kruskal-Wallis test.

With respect to comparison between drug groups, a total of 580 patients was required for greater than 85% power in detecting a 9% absolute difference, 0.89 in terms of relative risk, between groups in the proportion of patients who attained at least 50% resolution of ST-segment elevation, which corresponds to the 50% previously observed absolute difference between abciximab and placebo,⁵ with a two-sided 2.5% significance level and an 85% expected event rate in the control group based on previous findings.⁵ The noninferiority test was computed with the continuity-corrected χ² of Dunnett and Gent on the entire patient cohort. This was based on both intention-to-treat and per-protocol principles and was applied to an exploratory analysis across several prespecified subgroups. The Cochran-Mantel-Haenszel χ² test was performed to evaluate possible imbalances of the relative risk among different recruiting centers.

Tirofiban yielded noninferior recovery from ST-segment elevation after coronary intervention in comparison with abciximab; this result was consistent across different recruiting centers and multiple prespecified subgroups. Similarly, the rate of major adverse cardiovascular events (MACE, identified as the composite of death from any cause, reinfarction, and clinically-driven target-vessel revascularization) or bleeding events did not differ between the tirofiban or abciximab groups, but the incidence of severe or moderate thrombocytopenia was lower in the tirofiban group compared with the abciximab group, a finding of potential clinical relevance.¹⁹

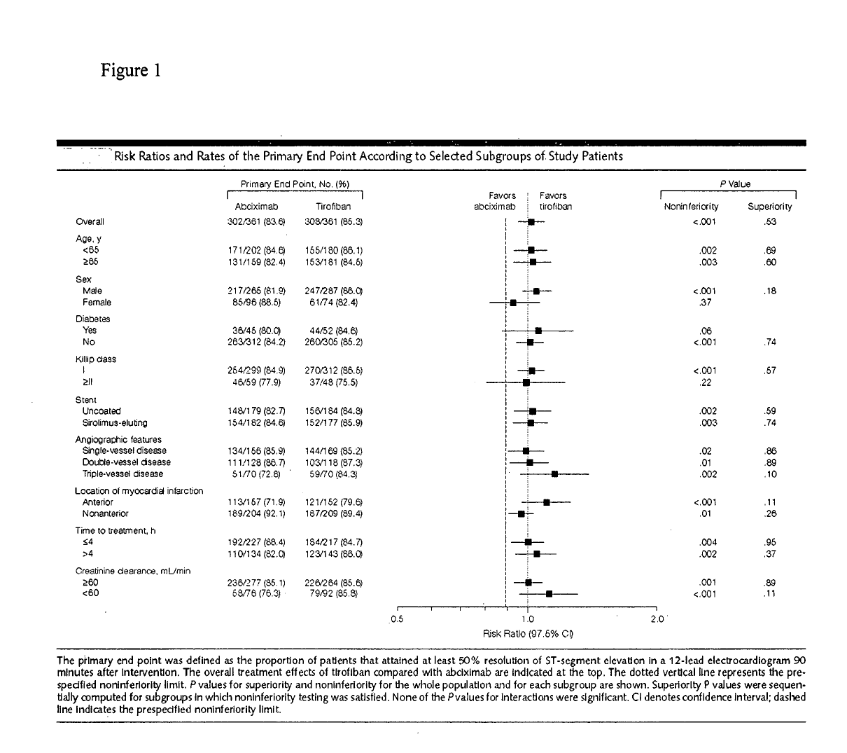

Normalization of ST-segment elevation is crucial for managing high-risk patients. ST-segment resolution, correlated to small infarct size and transmurality, is a strong and independent prognostic factor for death or death/MI (death or myocardial infarction), and internal controls from the MULTISTRATEGY study showed an increase in death/MI-free survival (95% versus 89%, P=0.023) for patients achieving ST-segment resolution of at least 50%. With respect to the comparison of HDB tirofiban and abciximab, the primary endpoint was the incidence of ≧50% resolution in ST-segment elevation within 90 minutes following percutaneous coronary intervention. The results of the study showed no significant difference in the percentage of patients achieving at least 50% ST-segment resolution between the abciximab (302 out of 361 patients, 83.6%) and the HDB-tirofiban-treated patients (308 out of 361 patients, 85.3%) in the intention-to-treat analysis (relative risk for tirofiban vs. abciximab, 1.020; 97.5% confidence interval, 0.958-1.086; P value<0.001 for noninferiority). The per-protocol analysis yielded similar results (relative risk, 1.020; 97.5% confidence interval, 0.959-1.086; P<0.001 for noninferiority). The data thus showed that treatment with HDB tirofiban led to noninferior ST-segment resolution compared with abciximab. (See FIG. 1.)

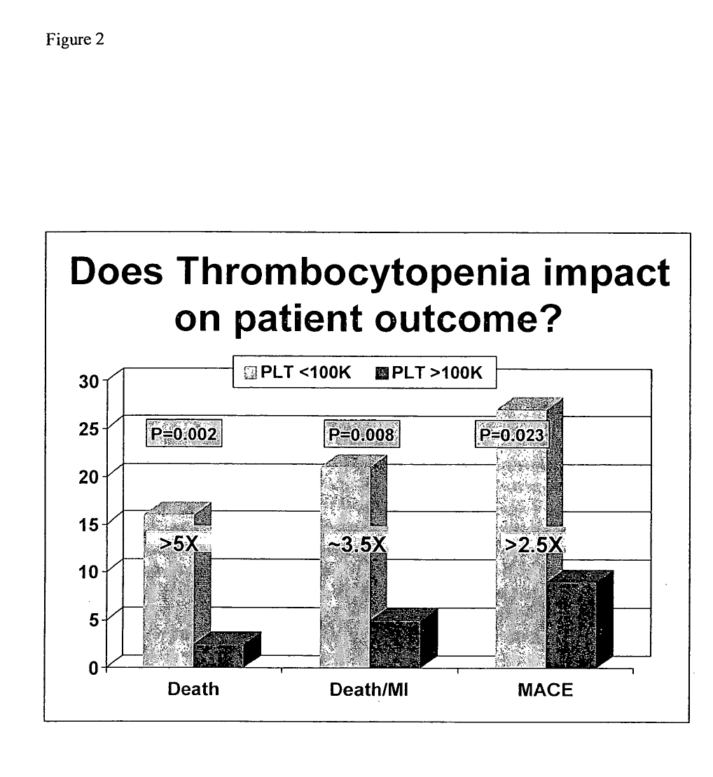

Surprisingly, the onset of thrombocytopenia had a significant effect on patient outcome. As can be seen in FIG. 2, patients with clinical thrombocytopenia (a platelet count of <100,000/μl [lighter shading]) were greater than five times more likely to die following the procedure than nonthrombocytopenic patients (platelet count of >100,000/μl [darker shading]). Similarly, thrombocytopenic patients were approximately three and one-half times more likely to die or have a myocardial infarction and greater than two and one-half times more likely to suffer a MACE following treatment than nonthrombocytopenic patients.

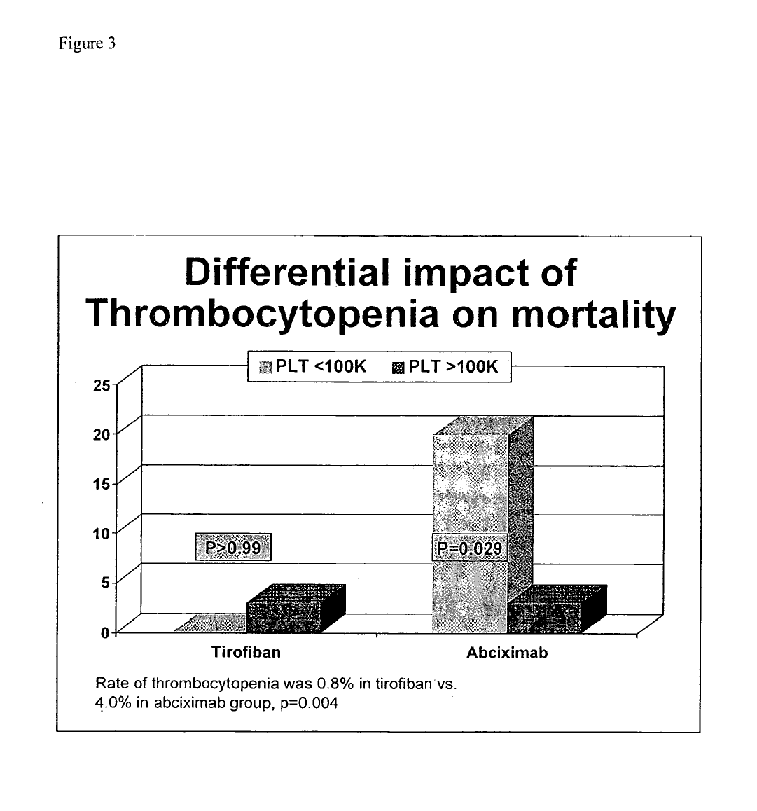

At 30 days, ischemic and hemorrhagic outcomes (Thrombolysis in Myocardial Infarction) (TIMI), major and minor bleeding) were similar in the HDB-tirofiban and abciximab groups (7.2% vs. 7.8%, P=0.89), as was the incidence of MACE (4.0% vs. 4.37%, P=0.85). However, the incidence of severe or moderate thrombocytopenia was found to be significantly greater in patients treated with abciximab compared with those treated with HDB tirofiban (4.0% vs. 0.8%, P=0.004). (See Table 1.) Even more striking, while the mortality rates of nonthrombocytopenic patients treated with HDB tirofiban or abciximab were virtually identical, a remarkable difference was seen in the follow-up study of the data in the mortality rate for patients who became thrombocytopenic following treatment with HDB tirofiban or abciximab. As can be seen in FIG. 3, 20% of the patients with abciximab-induced thrombocytopenia died within eight months, whereas none of the patients with HDB-tirofiban-induced thrombocytopenia died in the following eight-month period.

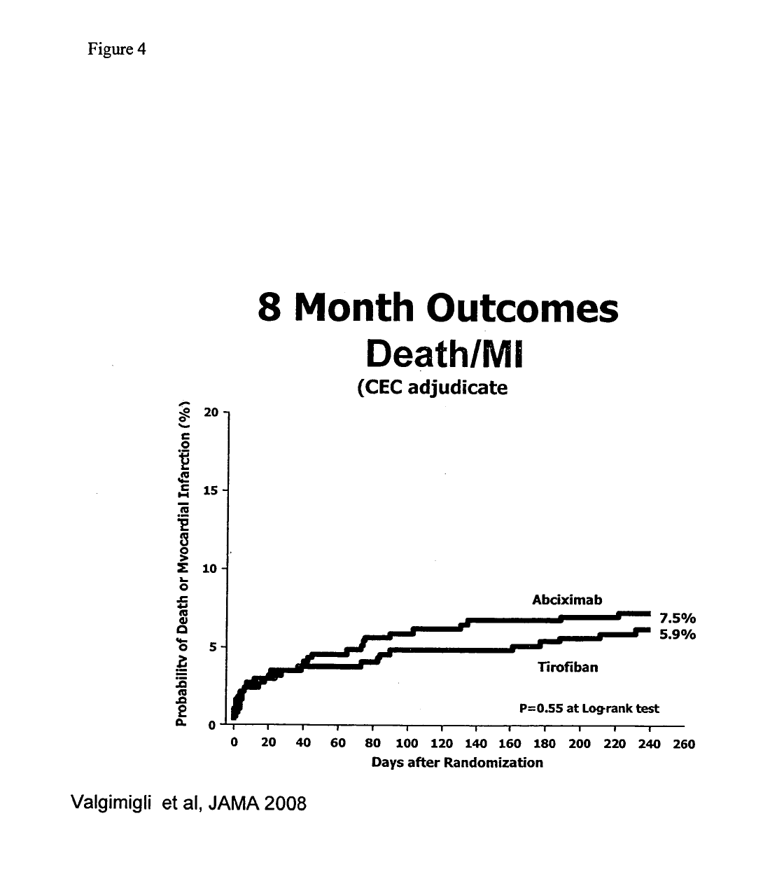

At eight months, the incidence of MACE was found to be similar between the HDB-tirofiban and abciximab treatment groups (9.9% vs. 12.4%, P=0.30) (Table 1). In the intent-to-treat population, the probability of death/MI within eight months after treatment was 7.5% for patients treated with abciximab versus 5.9% for patients treated with HDB tirofiban (P=0.55). (See Table 1 and FIG. 4.)

Thus, the data show in the first place that HDB-tirofiban treatment surprisingly results in significantly diminished incidence of severe or moderate thrombocytopenia compared to abciximab treatment. The data further show a surprising reduction in mortality of patients with HDB-tirofiban-induced thrombocytopenia vs. abciximab-induced thrombocytopenia.

TABLE 1
Kaplan-Meier Estimates of the Clinical Outcomes at 30 Days and 8 Months

| | Abciximab | Abciximab | Tirofiban | Tirofiban | Stents | | | | | |
|---|---|---|---|---|---|---|---|---|---|---|
| | Plus | Plus | Plus | Plus | | Sirolimus- | P Value | Drugs | | |
| | Uncoated | Sirolimus- | Uncoated | Sirolimus- | Uncoated | Eluting | Be- | | | P Value |
| | Stent | Eluting Stent | Stent | Eluting Stent | Stent | Stent | tween | Abciximab | Tirofiban | Between |
| Outcome | (n = 186) | (n = 186) | (n = 186) | (n = 186) | (n = 372) | (n = 372) | Stents | (n = 372) | (n = 372) | Drugs |

At 30 d, No. (%)

| | | | | | | | | | | | | | | | | | | |
|---|---|---|---|---|---|---|---|---|---|---|---|---|---|---|---|---|---|---|
| Death | 6 | (3.2) | 3 | (1.6) | 2 | (1.1) | 2 | (1.1) | 8 | (2.2) | 5 | (1.3) | .40 | 9 | (2.4) | 4 | (1.1) | .16 |
| Reinfarction | 5 | (2.7) | 0 | | 5 | (2.7) | 5 | (2.7) | 10 | (2.7) | 5 | (1.3) | .19 | 5 | (1.3) | 10 | (2.7) | .20 |
| Death or | 11 | (5.9) | 3 | (1.6) | 7 | (3.8) | 7 | (3.8) | 18 | (4.8) | 10 | (2.7) | .12 | 14 | (3.8) | 14 | (3.8) | .98 |
| reinfarction | | | | | | | | | | | | | | | | | | |
| Clinically driven | 5 | (2.7) | 1 | (0.5) | 3 | (1.6) | 5 | (2.7) | 8 | (2.2) | 6 | (1.8) | .59 | 6 | (1.6) | 8 | (2.2) | .59 |
| target-vessel | | | | | | | | | | | | | | | | | | |
| revascularization | | | | | | | | | | | | | | | | | | |
| Composite | 12 | (6.4) | 4 | (2.2) | 7 | (3.8) | 8 | (4.3) | 19 | (5.1) | 12 | (3.2) | .20 | 16 | (4.3) | 15 | (4.0) | .85 |
| of death, | | | | | | | | | | | | | | | | | | |
| reinfarction, | | | | | | | | | | | | | | | | | | |
| or target-vessel | | | | | | | | | | | | | | | | | | |
| revascularization | | | | | | | | | | | | | | | | | | |
| Definite stent | 4 | (2.2) | 1 | (0.5) | 3 | (1.6) | 4 | (2.2) | 7 | (1.9) | 5 | (1.3) | .56 | 5 | (1.3) | 7 | (1.9) | .56 |
| thrombosis | | | | | | | | | | | | | | | | | | |
| Probable stent | 2 | (1.1) | 1 | (0.5) | 2 | (1.1) | 0 | | 4 | (1.1) | 1 | (0.3) | .18 | 3 | (0.8) | 2 | (0.5) | .65 |
| thrombosis | | | | | | | | | | | | | | | | | | |
| Definite or | 6 | (3.2) | 2 | (1.1) | 5 | (2.7) | 4 | (2.2) | 11 | (3.0) | 6 | (1.6) | .22 | 8 | (2.2) | 9 | (2.4) | .81 |
| probable stent | | | | | | | | | | | | | | | | | | |
| thrombosis | | | | | | | | | | | | | | | | | | |
| Safety analysis | | | | | | | | | | | | | | | | | | |
| Major bleeding | 3 | (1.6) | 3 | (1.6) | 5 | (2.7) | 4 | (2.2) | 8 | (2.2) | 7 | (1.9) | .79 | 6 | (1.6) | 9 | (2.4) | .44 |
| Minor bleedng | 15 | (8.1) | 8 | (4.3) | 11 | (5.9) | 7 | (3.8) | 26 | (7.0) | 15 | (4.0) | .09 | 23 | (6.2) | 18 | (4.8) | .40 |
| Red blood cells | | | | | | | | | | | | | | | | | | |
| transfusion | | | | | | | | | | | | | | | | | | |
| ≧1 Units | 4 | (2.2) | 4 | (2.2) | 9 | (4.8) | 5 | (2.7) | 13 | (3.5) | 9 | (2.4) | .39 | 8 | (2.2) | 14 | (3.8) | .20 |
| ≧2 Units | 4 | (2.2) | 4 | (2.2) | 6 | (3.2) | 3 | (1.6) | 10 | (2.7) | 7 | (1.9) | .46 | 8 | (2.2) | 9 | (2.4) | .82 |
| Severe | 6 | (3.2) | 3 | (1.6) | 2 | (1.1) | 0 | | 8 | (2.2) | 3 | (0.8) | .23 | 9 | (2.4) | 2 | (0.5) | .03 |
| thrombocytoperia | | | | | | | | | | | | | | | | | | |
| (<50000 | | | | | | | | | | | | | | | | | | |
| cells/mm³) | | | | | | | | | | | | | | | | | | |
| Moderate | 2 | (1.1) | 4 | (2.2) | 1 | (0.5) | 0 | | 3 | (0.8) | 4 | (1.1) | .70 | 6 | (1.6) | 1 | (0.3) | .06 |
| thrombocytoperia | | | | | | | | | | | | | | | | | | |
| (<100000 | | | | | | | | | | | | | | | | | | |
| cells/mm³) | | | | | | | | | | | | | | | | | | |

At 8 mo, No. (%)

| | | | | | | | | | | | | | | | | | | |
|---|---|---|---|---|---|---|---|---|---|---|---|---|---|---|---|---|---|---|
| Composite of | 30 | (16.1) | 16 | (8.6) | 24 | (12.9) | 13 | (7.0) | 54 | (14.5) | 29 | (7.8) | .004 | 46 | (12.4) | 37 | (9.9) | .30 |
| death, | | | | | | | | | | | | | | | | | | |
| reinfarction, | | | | | | | | | | | | | | | | | | |
| or target-vessel | | | | | | | | | | | | | | | | | | |
| revascularization | | | | | | | | | | | | | | | | | | |
| Death | 8 | (4.3) | 7 | (3.8) | 7 | (3.8) | 4 | (2.2) | 15 | (4.0) | 11 | (3.0) | .42 | 15 | (4.0) | 11 | (3.0) | .42 |
| Reinfarction | 9 | (4.8) | 4 | (2.2) | 8 | (4.3) | 8 | (4.3) | 17 | (4.6) | 12 | (3.2) | .34 | 13 | (3.5) | 16 | (4.3) | .57 |
| Death or | 16 | (8.6) | 11 | (5.9) | 12 | (6.5) | 11 | (5.9) | 28 | (7.5) | 22 | (5.9) | .37 | 28 | (7.5) | 22 | (5.9) | .55 |
| reinfarction | | | | | | | | | | | | | | | | | | |
| Clinically driven | 21 | (11.3) | 6 | (3.2) | 17 | (9.1) | 6 | (3.2) | 38 | (10.2) | 12 | (3.2) | <.001 | 27 | (7.3) | 23 | (6.2) | .58 |
| target-vessel | | | | | | | | | | | | | | | | | | |
| revascularization | | | | | | | | | | | | | | | | | | |
| Definite stent | 7 | (3.8) | 3 | (1.6) | 4 | (2.2) | 6 | (3.2) | 11 | (3.0) | 9 | (2.4) | .65 | 10 | (2.7) | 10 | (2.7) | .99 |
| thrombosis | | | | | | | | | | | | | | | | | | |
| Possible stent | 1 | (0.5) | 3 | (1.6) | 3 | (1.6) | 0 | | 4 | (1.1) | 3 | (0.8) | .71 | 4 | (1.1) | 3 | (0.8) | .70 |
| thrombosis | | | | | | | | | | | | | | | | | | |
| Definite or | 9 | (4.8) | 4 | (2.2) | 6 | (3.2) | 6 | (3.2) | 15 | (4.0) | 10 | (2.7) | .31 | 13 | (3.5) | 12 | (3.2) | .85 |
| probable stent | | | | | | | | | | | | | | | | | | |
| thrombosis | | | | | | | | | | | | | | | | | | |
| Definite or | 9 | (4.8) | 7 | (3.8) | 8 | (4.3) | 6 | (3.2) | 17 | (4.6) | 13 | (3.5) | .45 | 16 | (4.3) | 14 | (3.8) | .71 |
| probable or | | | | | | | | | | | | | | | | | | |
| possible stent | | | | | | | | | | | | | | | | | | |
| thrombosis | | | | | | | | | | | | | | | | | | |

The methods of the present invention can be employed during the treatment of any patients for whom inhibition of platelet aggregation or adhesion is desired or required. Such patients can include patients who are already thrombocytopenic, are prethrombocytopenic or predisposed to thrombocytopenia, or are normal in this regard. The treatments to which the patients are being subjected may be, but are not confined to, arterial grafts, carotid endaterectomy and other cardiovascular procedures wherein manipulation of arteries or organs, and/or the interaction of platelets with artificial surfaces, leads to platelet aggregation and potential formation of thrombi and thromboemboli.

The practice of the invention is not limited to the preferred administration regimen described earlier herein; any suitable HDB/continuous-infusion regimen may be employed. For example, the HDB may be in the range of about 20 to about 30 μg/kg and the subsequent continuous infusion may be in the range of about 0.10 to about 0.20 μg/kg/min for a period of about 6 to about 108 hours.

The practice of the invention is not limited to the administration of the hydrochloride salt of tirofiban; any pharmaceutically acceptable tirofiban salt may be employed. Such salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylbromide, methyInitrate, methylsulfate, mucate, napsylate, nitrate, oleate, oxalate, pamoate, palmitate, panthothenate, phosphate/diphosphate, polygalacturonate, salicylate, stearate, subacetate, succinate, tannate, tartrate, teoclate, tosylate, triethiodide and valerate.

### REFERENCES

1. Frossard M, Fuchs I, Leitner J M, et al. Platelet function predicts myocardial damage in patients with acute myocardial infarction. Circulation. 2004; 110(11): 1392-1397.
2. Campo G, Valgimigli M, Gemmati D, et al. Value of platelet reactivity in predicting response to treatment and clinical outcome in patients undergoing primary coronary intervention: insights into the STRATEGY Study. J Am Coll Cardiol. 2006; 48(11): 2178-2185.
3. Huczek Z, Filipiak K J, Kochman J, et al. Baseline platelet reactivity in acute myocardial infarction treated with primary angioplasty: influence on myocardial reperfusion, left ventricular performance, and clinical events. Am Heart J. 2007; 154(1): 62-70.
4. Antoniucci D, Migliorini A, Parodi G, et al. Abciximab-supported infarct artery stent implantation for acute myocardial infarction and long-term survival: a prospective, multicenter, randomized trial comparing infarct artery stenting plus abciximab with stenting alone. Circulation. 2004; 109(14): 1704-1706.
5. Antoniucci D, Rodriguez A, Hempel A, et al. A randomized trial comparing primary infarct artery stenting with or without abciximab in acute myocardial infarction. J Am Coll Cardiol. 2003; 42(11): 1879-1885.
6. Topol E J, Byzova T V, Plow E F. Platelet GPIIb-IIIa blockers. Lancet. 1999; 353(9148): 227-231.
7. Lele M, Sajid M, Wajih N, Stouffer G A. Eptifibatide and 7E3, but not tirofiban, inhibit alpha(v) beta(3) integrin-mediated binding of smooth muscle cells to thrombospondin and prothrombin. Circulation. 2001; 104(5): 582-587.
8. Reininger A J, Agneskirchner J, Bode P A, Spannagl M, Wurzinger L J. c7E3 Fab inhibits low shear flow modulated platelet adhesion to endothelium and surface-absorbed fibrinogen by blocking platelet GP IIb/IIIa as well as endothelial vitronectin receptor: results from patients with acute myocardial infarction and healthy controls. Thromb Haemost. 2000; 83(2): 217-223.
9. Topol E J, Moliterno D J, Herrmann H C, et al. Comparison of two platelet glycoprotein IIb/IIIa inhibitors, tirofiban and abciximab, for the prevention of ischemic events with percutaneous coronary revascularization. N Engl J. Med. 2001; 344(25): 1888-1894.
10. Schneider D J, Herrmann H C, Lakkis N, et al. Increased concentrations of tirofiban in blood and their correlation with inhibition of platelet aggregation after greater bolus doses of tirofiban. Am J. Cardiol. 2003; 91(3): 334-336.
11. Valgimigli M, Percoco G, Malagutti P, et al. Tirofiban and sirolimus-eluting stent vs abciximab and bare-metal stent for acute myocardial infarction: a randomized trial. JAMA. 2005; 293(17): 2109-2117.
12. Danzi G B, Capuano C, Sesana M, Mauri L, Sozzi F B. Variability in extent of platelet function inhibition after administration of optimal dose of glycoprotein IIb/IIIa receptor blockers in patients undergoing a high-risk percutaneous coronary intervention. Am J. Cardiol. 2006; 97(4): 489-493.
13. Ernst N M, Suryapranata H, Miedema K, et al. Achieved platelet aggregation inhibition after different antiplatelet regimens during percutaneous coronary intervention for ST-segment elevation myocardial infarction. J Am Coll Cardiol. 2004; 44(6): 1187-1193.
14. Bolognese L, Falsini G, Liistro F, et al. Randomized comparison of upstream tirofiban versus downstream high bolus dose tirofiban or abciximab on tissue-level perfusion and troponin release in high-risk acute coronary syndromes treated with percutaneous coronary interventions: the EVEREST trial. J Am Coll Cardiol. 2006; 47(3): 522-528.
15. Danzi G B, Sesana M, Capuano C, Mauri L, Berra Centurini P, Baglini R. Comparison in patients having primary coronary angioplasty of abciximab versus tirofiban on recovery of left ventricular function. Am J Cardiol. 2004; 94(1): 35-39.
16. TENACITY trial officially halted; Guilford seeks partner or buyer for tirofiban. http://www.theheart.org/viewArticle.do?primaryKey=516083&from=/searchLayout.do. Accessed Jan. 9, 2008.
17. Valgimigli M, Bolognese L, Anselmi M, et al. Two-by-two factorial comparison of high-bolus-dose tirofiban followed by standard infusion versus abciximab and sirolimus-eluting versus bare-metal stent implantation in patients with acute myocardial infarction: design and rationale for the MULTI-STRATEGY trial. Am Heart J. 2007; 154(1): 39-45.
18. Cutlip D E, Windecker S, Mehran R, et al. Clinical end points in coronary stent trials: a case for standardized definitions. Circulation. 2007; 115(17): 2344-2351.
19. Merlini P A, Rossi M, Menozzi A, et al. Thrombocytopenia caused by abciximab or tirofiban and its association with clinical outcome in patients undergoing coronary stenting. Circulation. 2004; 109(18): 2203-2206.

### FIGURE LEGENDS

FIG. 1: A noninferiority analysis of tirofiban with respect to abciximab is shown. The analysis is based on comparison of effectiveness in achieving the primary end point, defined as attainment of at least 50% resolution of/recovery from ST-segment elevation in a 12-lead electrocardiogram 90 minutes after intervention.

FIG. 2: A comparison is shown of the outcome within eight months for patients treated for myocardial infarction depending on whether or not they become thrombocytopenic (lighter shading vs. darker shading) as a side effect of the treatment. Compared are the chances for death from any cause; for death or another myocardial infarction; and for occurrence of a major adverse cardiovascular event (defined as the composite of death from any cause, reinfarction, and clinically-driven target-vessel revascularization within the first eight months).

FIG. 3: A comparison is shown of the chances of mortality within eight months of treatment in myocardial-infarction patients treated either with HDB tirofiban or abciximab and depending on whether the patients were thrombocytopenic (lighter shading) or not (darker shading).

FIG. 4: A comparison is shown of the probability of experiencing a clinical event (death or reinfarction) within eight months of treatment of myocardial-infarction patients treated either with HDB tirofiban or abciximab.

We claim:

1. A method for reducing the risk of thrombocytopenia-associated mortality which comprises administering to a patient suffering from a condition whose treatment requires the inhibition of platelet aggregation a therapeutically effective amount of a pharmaceutically acceptable salt of tirofiban.

2. A method for reducing the risk of becoming thrombocytopenic which comprises administering to a patient suffering from a condition whose treatment requires the inhibition of platelet aggregation a therapeutically effective amount of a pharmaceutically acceptable salt of tirofiban.

3. A method for reducing the risk of thrombocytopenia-associated morbidity which comprises administering to a patient suffering from a condition whose treatment requires the inhibition of platelet aggregation a therapeutically effective amount of a pharmaceutically acceptable salt of tirofiban.

4. The method according to any one of claims 1-3, wherein the tirofiban is administered intravenously as a high-dose bolus followed by a continuous infusion over time.

5. The method according to claim 4, wherein the high-dose bolus is about 25 μg/kg and the continuous infusion is about 0.15 μg/kg/min for about 18-24 hours.

6. The method according to any one of claims 1-3, wherein tirofiban hydrochloride is administered.

7. The method according to claim 4, wherein tirofiban hydrochloride is administered.

8. The method according to claim 5, wherein tirofiban hydrochloride is administered.

9. The method according to claim 1 or 3, wherein the patient is already thrombocytopenic; prethrombocytopenic or predisposed to thrombocytopenia; or normal in this regard.
